(19) United States
(12) Patent Application Publication
(10) Pub. No.: US 2014/0228661 A1
(43) Pub. Date: Aug. 14, 2014

(54) BIOPSY METHOD AND GUN SET DEVICES

(71) Applicants: Liviu Popa-Simil, Los Alamos, NM (US); Radu Kramer, Woodcliff Lake, NJ (US)

(72) Inventors: Liviu Popa-Simil, Los Alamos, NM (US); Radu Kramer, Woodcliff Lake, NJ (US)

(21) Appl. No.: 14/164,762

(22) Filed: Jan. 27, 2014

Related U.S. Application Data
(60) Provisional application No. 61/762,582, filed on Feb. 8, 2013.

(51) Int. Cl. A61B 10/02; A61B 5/145; A61B 5/00; A61B 5/06

(52) U.S. Cl. CPC A61B 10/0275; A61B 10/0283; A61B 5/061; A61B 5/14539; A61B 5/0075
USPC 600/361; 600/424

(57) ABSTRACT

A novel set of biopsy-gun related tools are developed in order to make a better safer sampling and diagnosis, comprising a set of tools that takes the sample, releases a drug impregnated plug and additionally may inject drugs, measure pH and the molecular content of the penetrated tissues.

A novel technology aiming to minimize the penetrated tissue damage, using a small diameter needle with capabilities for immediate spectroscopic analysis of the tissue, and followed by sampling and plugging of the tissue when needed.

The invention describes a set of variable diameter thin tubes used to guide and insulate the penetrated organs, and a final operation of plugging the wounds with absorbable substances, impregnated with drugs.

The supplementary low intensity radiation sources could increase the processes' accuracy and safety, offering the opportunity of gathering supplementary x-densitometry information.

### CROSS REFERENCE TO RELATED APPLICATIONS

This application claims the benefit of U.S. Provisional Application No. 61/762,582 from Feb. 08, 2013, and no international application.

### BACKGROUND

A biopsy is a medical procedure to acquire cells or tissues for pathological examination. It normally involves the removal of tissue from a living subject to determine the presence or extent of disease. The tissue is generally examined under microscopy by a pathologist, and can also be analyzed chemically. When an entire lump or suspicious area is removed, the procedure is called an excisional biopsy. When only a sample of tissue is removed with preservation of the histological architecture of the tissue's cells, the procedure is called an incisional biopsy or core biopsy. When a sample of tissue or fluid is removed with a needle in such a way that cells are removed without preserving the histological architecture of the tissue cells, the procedure is called a needle aspiration biopsy.

The actual tissue sampling is often done percutaneously with a long and fairly large bore needle designed for tissue removal, attached to a syringe or a more elaborate apparatus called a biopsy gun. The needle apparatus is usually passed several times through the tissue to remove several tissue samples. Percutaneous needle biopsies are often done using x-ray (usually CT) or ultrasound, to guide the surgeon to the right area.

An open biopsy is a surgical procedure, done in an operating room or outpatient surgical area, using local or general anesthesia. The surgeon cuts into the organ being sampled, and removes tissue under direct visualization using a needle or excision.
Closed endoscopic biopsy uses a much smaller surgical cut than open biopsy. A small cut is made so that a camera-like instrument attached to a flexible or rigid hollow tubing can be inserted, guided to the organ in question, and samples taken by needle or small cutting devices operated remotely.

There are known biopsy devices for use with Bard® MAGNUM® and BIP High Speed Multi Biopsy Instrument. High quality core biopsy needles provide histological information for diagnostic characterization of suspect lesions.
Unique, patented hub design obsoletes needle “spacers”. A 19 mm sample notch ensures sufficient tissue for clinical diagnosis.
Each needle has an echogenic tip, promoting accurate placement under ultrasound guidance.
Numerically ordered markings facilitate precise depth placement.
Hubs are color coded for easy gauge size determination, available in a variety of gauge sizes and lengths.
Needles are color coded for easy gauge size determination, which can be seen through the “window” on the bottom of the device.
Can be used with Co-axial Introducer Needles.
Small instrument size makes the Tru-Core® II compatible with most upright stereo- tactic machines.
Simple “pull/push” operation of the control knobs creates a device which is truly single-handed in operation. The Tru-Core® II can easily be operated with one hand making it ideal for procedures requiring ultrasound guidance. Its small size and lightweight make it equally ideal for CT guided core biopsies.
A 22 mm “throw” advancement, combined with a 19 mm sample notch, harvest sufficient tissue for clinical diagnosis

U.S. Pat. No. 5,195,988 is teaching that a hemostatic gelatin sheath is fitted as a portion of an outer cannula over the distal portion of the inner cutting end of a biopsy needle, with the goal of preventing leakage of material from the organ or surrounding tissue. When positioned properly, the cannulas accurately deposit the hemostatic sheath at the position where the biopsy specimen was taken, and the needle with the specimen within it is then withdrawn. The in-situ sheath minimizes bleeding from the biopsy site before the body absorbs the gelatin.

More, the U.S. Pat. No. 4,838,280 is teaching a similar hemostatic gelatin sheath which is fitted as a portion of an outer cannula over the distal portion of the inner cutting end of a biopsy needle. The hemostatic sheath is positioned exactly where the biopsy specimen was taken and the needle with the specimen within it is then withdrawn. The sheath remaining in situ minimizes bleeding from the biopsy site as the body absorbs the gelatin.

These ideas, which comprise the previous art, have evident difficulties with the possible misplacement of the outer cannula as it penetrates to the biopsy site, as well as failure to be located exactly at the right location to prevent bleeding. These problems are solved by the present invention.

In the U.S. Pat. No. 5,080,655 an improved medical needle is disclosed which has a bio-absorbable gelatin cutting or puncturing tip formed therein. The gelatin feature renders the needle incapable of penetration after one use. One problem with this tip is that it is not sharp enough, so its failure to penetrate may cause more complications. Additionally, the gelatin partially dissolves to leave a coating on the punctured tissue margin which acts to minimize hemorrhaging complications. But if the gelatin is deposited prematurely before it reaches the biopsy site it cannot prevent bleeding from the biopsy site. Hemorrhaging complications are alternatively addressed by a non-bio-absorbable sheath left in-situ positioned at the biopsy site which compresses the tissue. The problems described here are corrected by the present invention.

U.S. Pat. No. 4,785,826 relates to an instrument known variously as biopsy needle or cannula and used to gather tissue, and particularly soft tissue such as bone marrow, from living persons or animals for pathological study. The instrument retains the sampled tissue specimen by closing the end of a hollow cannula while the end is still in the sampling position, and more particularly by deforming a flexible portion of the inner sampling shaft. This device has one moving part within the other. A flexible portion of the inner part is displaced or deformed to occlude an open tissue, receiving end thereof and thereby capture tissue and retain it against loss on removal of the instrument from the tissue into which it has been thrust. This device has some operational problems, as interaction with various hardness and viscosity of organs provides various volumes of biopsy tissue. This impediment is corrected by the present patent that assures about same predictable amount of tissue with minimum organ damage.

The U.S. Pat. No. 5,061,281 teaches an implantable medical device capable of encouraging cellular growth and regeneration of function fabricated totally or in part from one or more bioresorbable polymers, as for example bioresorbable homopolymers derived from the polymerization of alpha-hydroxy-carboxylic acids, where at least one of the polymers has an average molecular weight of from about 234,000 to about 320,000 as measured by gel permeation chromatography.

The U.S. Pat. No. 4,749,689 relates to a hemostatic agent used in surgical operations, which can be produced in two ways: one blending collagen/gelatin with protamine, the other blending collagen/gelatin with protamine and a bi-functional cross-linking agent so as to make said collagen/gelatin have a covalent bond with said protamine. The produced hemostatic' agent can stop bleeding within far less time than a conventional hemostatic agent made out of pure collagen.

The U.S. Pat. No. 4,412,947 is teaching a process for preparing a coherent porous collagen sheet material, comprised of forming natural insoluble particulate collagen in substantially pure form and suspending the particulate collagen in a weak aqueous organic acid solution while maintaining the collagen in particulate form. The suspension is freeze-dried to form a coherent porous native collagen sheet material which is useful as a wound dressing, burn dressing, hemostatic sheet or the like. The present invention makes use of all these developments, to better serve the cause of hemostasis and/or organ regeneration.

The research in cell and tissue analysis using a multi-modal infra red (IR) microscopy, as by GD Sockalingum, may be incorporated in the present method. For more than a decade, the possibilities of IR spectroscopy have been explored to distinguish different biomolecules by probing chemical bond vibrations and using these molecular and submolecular patterns to define and differentiate between normal and diseased states. IR spectroscopy provides a spectral signature of the intensity and a spatial location of the chemical components, thus highlighting biochemical changes. Studies on biological specimens (fluids, cells and tissues) have been carried out either with instruments employing bench-top light sources or, more recently, with spectrometers using multidetector devices or synchrotron radiation sources; the two latter methods show an improvement in spectral quality and acquisition time. These tools have opened new potential frontiers in biomedical research, affording results unavailable by conventional cyto- and histo-pathology.

This invention uses the potential of IR-based mapping and imaging, a fast emerging biophotonic technology, for investigating cells and tissues.

In the case of single cell analysis, others have demonstrated feasibility with synchrotron-IR sources and UV, Vis IR tunable lasers as well with IR spectral detections. These high resolution measurements offer new possibilities for intra- and pericellular analysis, allowing one to determine the bio-distribution of intrinsic molecules of interest (proteins, nucleic acids, lipids) in a non-invasive manner without any staining. Compared to biomarker-based approaches, which only detect selected molecules, IR spectrum analysis can detect a wide range of molecules and can more accurately characterize the whole cell under investigation. The possible use of IR spectroscopy for monitoring cell-drug interaction and for a deeper understanding of cell migration is also approached by the present invention.

In a paper published in Annu. Rev. Phys. Chem. 1996. 47:555-606 entitled “Quantitative Optical Spectroscopy For Tissue Diagnosis” the authors show that the interaction of light with tissue has been used to recognize disease since the mid-1800s. The recent developments of small light sources, detectors, and fiber optic probes provide opportunities to quantitatively measure these interactions, which yield information for diagnosis at the biochemical, structural, or (patho)physiological level within intact tissues. However, because of the strong scattering properties of tissues, the reemitted optical signal is often influenced by changes in biochemistry (as detected by these spectroscopic approaches) and by physiological and pathophysiological changes in tissue scattering. One challenge of biomedical optics is to uncouple the signals influenced by biochemistry, which themselves provide specificity for identifying diseased states, from those influenced by tissue scattering, which are typically not specific to a pathology. Therefore it is important to understand optical signal interactions (fluorescence, fluorescence lifetime, phosphorescence, and Raman with cells, cultures, and tissues) and then provide a descriptive framework for light interaction based upon tissue absorption and scattering properties, and important endogenous and exogenous biological chromophores in order to employ these signals for detection and diagnosis of disease.

Further scientific work was related to Fiber-optic Probes for Mid-infrared Spectrometry. Peter J. Melling and Mary Thomson from Remspec Corporation, Sturbridge, Mass., USA state that chemical composition sensors incorporated in mid-infrared fiber-optic probes are commercially available and provide a wide range of capability. Basically if a technique can be used in the sample compartment there is a fiber-optic equivalent available. Fiber-optic techniques are quantitative and can almost always be calibrated. This combined with the flexibility and ability to measure in situations where taking a sample is not possible, means that fiber optics provides a very powerful technique to analytical chemists.

The mid-infrared region of the spectrum is 4000 cm⁻¹ to 400 cm⁻¹ (2.5 μm to 25 μm). In that range occur most of the fundamental molecular vibrations and many of the first overtones and combinations. The bands in the mid-infrared tend to be sharp and have very high absorptivity, both characteristics being desirable. Because the bands are sharp, most small molecules have distinctive spectral “fingerprints” that can be readily identified in mixtures. Also, because individual peaks can often be associated with particular functional groups, it is possible to see changes in the spectrum of an individual reagent due to a specific chemical reaction.

The Japanese patent 3566232 and U.S. Pat. No. 5,995,696, GB 2361776 teaches that Laser light of the wavelength of 2 μm or longer in the infrared region is useful for medical and industrial processing applications, and sources for such light include the Er-YAG and CO₂ lasers. In this wavelength region, silica glass fiber optics cannot be used for delivering such light. The flexible hollow fibers for infrared laser light transmission have a wide band transmittance over 90% and may be used with minor absorption up to 50 microns or more behaving as the EM wave-guides. As the core region is hollow, the damage threshold of the fiber end face is high, and thus, the newly developed fibers are suitable for high power laser energy delivery.

The fibers are not only effective for invisible infrared light, but visible light can also be superposed for visualization and guiding purposes; and air can also be introduced through the fibers. These hollow fibers have made it possible to construct laser systems with higher operability than conventional articulated arm delivery systems consisting of minors and arms. The present patent uses these developments in optical spectroscopy to create a more advanced approach to minimally invasive disease diagnosis.

### SUMMARY

The present invention discloses a novel method to make tissue diagnosis that relies on a new set of diagnostic needles. The method consists in using a very thin optical needle and sheathing, making a very small hole of 1 mm or less, advancing slowly towards the diseased organ, while measuring the molecular composition of the tissues in the path. If the data obtained is not conclusive, a larger sheath and then a biopsy needle is introduced.

The biopsy needle may also contain measurement and visualization systems but is designed to cut a tissue sample and extract it for pathological diagnosis by conventional means. After taking the sample a plug is released in place with hemo-cito- static properties, applying the first cure to the sick tissue. After the biopsy needle is withdrawn a specialized plugging needle is introduced through the sheath, and each penetrated tissue surface is plugged with appropriate substance and the sheath gradually withdrawn leaving the patient minimally damaged. All the needles moving in and out from the body is pressure assisted introducing compensating fluids to prevent the creation of negative pressures that may accelerate leakage and inter-contamination of the internal organs.

A novel TISSUE BIOPSY needle conceived to minimize the effects of tissue penetration and rupture by dropping potential tumor customized drug impregnated plugs in order to cure and stop inter-tissue exchanges and seal the penetration. The plugs may have a specialized shape that anchor in the tissue and seals the plug, releasing its drug, being resorbable and fully compatible with the tissue.

A needle that is protected by several sheaths one for each suspected unhealthy penetrated tissue in order to reduce inter-contamination and the spread of the sick cells. A sample notch is supported on an internal rod, that on the opposite side has a drug impregnated plug that is released simultaneously with the tissue harvesting process. The process is enhanced by the use of a capillary tube that brings negative pressure in the sample notch fixing the sample on the pad in order to be cut smoothly, without generating shearing in the tissue, while a positive pressure works synchronous with the cutter tube hook that detaches the drug-plug that will expand in the tissue as soon as the tube is withdrawn.

Using exchangeable cores the sample cutter may be extracted and a new tube may be introduces that delivers specialized plugs along the penetration hole, minimizing the penetration damage to the various tissues penetrated. One of the sampler tips or a specialized penetration tip, may be provided with a set of radioactive point sources operating on different energies and providing a stereoscopic view from inside the body, simultaneously with an accurate position and direction, making it redundant to the ultrasound system frequently used, and providing a coordinate localization of all operations. A set of hollow-capillary optic fiber is passing through the tube allowing a complex optical UV and IR spectroscopy in order to map the composition of the penetrated tissues, and could even resonantly damage the selected molecular bonds in the ill cells, killing the tumor.

The entire system, a multi-gun device, is held on a robotic arm with computerized coordinate control in order to minimize the damage. The gun holder has a dynamic pressure control in order to stop the liquid penetration and interchange.

### BRIEF DESCRIPTION OF THE DRAWINGS

FIG. 1A—Present art BIOPSY needle open for sampling, longitudinal section.

FIG. 1B—Present art sampler needle closed after sampling, longitudinal section.

FIG. 1C—Present art sampler needle open for sampling, longitudinal section with diameter zoomed by a factor of 4.

FIG. 1D—Present art sampler needle closed sampling, longitudinal section with diameter zoomed by a factor of 4.

FIG. 1E—Cross section through AA′ plane in FIG. 1D.

FIG. 1F—Cross section through BB′ plane in FIG. 1D.

FIG. 1G—Cross section through CC′ plane in FIG. 1D.

FIG. 2A—Sampler in the tissue in open position, ready to cut.

FIG. 2B—Sampler in tissue in closed position after cutting the tissue.

FIG. 2C—Cross section AA′ in FIG. 2B, showing the plug outside the needle.

FIG. 2D—Front view of the front of the sampling needle.

FIG. 3A—Needle with supplementary tube.

FIG. 3B—Cross section of the sampling Needle in AA′ position.

FIG. 3C—Obtaining tissue for pathology, with other sheath over biopsy gun in position before penetrating the tissue.

FIG. 3D—Obtaining tissue for pathology, with other sheath over biopsy gun with biopsy gun extended in the tissue.

FIG. 3E—Obtaining tissue for pathology, with other sheath over biopsy gun with the biopsy gun with the tissue specimen loaded.

FIG. 4A—Longitudinal section through the needle with profiled cutting head.

FIG. 4B—Front view of the needle with profiled cutting head.

FIG. 4C—Tissue scanner with cyto-hemo-static biopsy gun, empty sheath.

FIG. 4D—Tissue scanner with hollow-core optic fibers.

FIG. 4E—Tissue scanner with a plurality of hollow core optic fibers.

FIG. 4F—Tissue scanner front view of the tip.

FIG. 5A—Longitudinal section of the needle with inter-organ plug releaser.

FIG. 5B—Cross section AA′ in FIG. 5A through the piston for organ interface plug releaser piston.

FIG. 5C—The longitudinal section for the introduction of cyto-hemo-static material using the protective sheath at the beginning of range.

FIG. 5D—The longitudinal section for the introduction of cyto-hemo-static material using the protective sheath, ready to implant in the tissue.

FIG. 5E—The longitudinal section for the introduction of cyto-hemo-static material using the protective sheath implanted in the targeted tissue.

FIG. 5F—A simplistic version of application of the method using an ultrasound probe and the sheath with either scanner or biopsy gun.

FIG. 6A—Schematic view of an abdominal biopsy-sampling needle inserting process.

FIG. 6B—Schematic view of an abdominal biopsy-sampling needle in the sampling position.

FIG. 6C—Schematic view of an abdominal biopsy-sampling after the needle was extracted and the wound plugged.

FIG. 7—Schematic view of an abdominal biopsy biopsy-sampling using radio-goniometry.

FIG. 8—Schematic view of the capillary-imaging device.

### DETAILED DESCRIPTION OF THE INVENTION

The inventors consider that most of the problems generated by the actual biopsy operations are both due to the obsolete procedure and equipment used to sample the patient's tissue and due to the penetration tool leaving a large communication hole through the internal organs where infections may propagate. Therefore we develop a new method of penetration with a more complex tool that uses a gradual approach inside sheathed tubes that first aims for very small perforation using liquid injection, in order to reduce the stress and tissue stretching as much as possible. The assembly comes as the a set of instruments to assure the gradual penetration of the ultra thin optical investigation needle with appropriate sheathing and plugging, optionally followed by thicker gauge biopsy needles with appropriate sheathing and plugging tools, with dynamic pressure compensation systems to prevent undesired pressures developing.

The method is straightforward, and starts with coordinates localization of the organ of interest. The best access route is established and the thin gauge needle with hollow capillary for optical analysis is gradually inserted, together with the sheath. As the needle is inserted, analysis is performed in order to identify diseased cells and molecules and measure their concentration. If this investigation result is good enough, the gauge is slowly withdrawn leaving behind a liquid with hemo-static properties that may contain a drug if desired. If the diagnosis is not clear enough at this point to make a good treatment decision, and the tissue biopsy is required, the sheath diameter is gradually enlarged until sufficient for the biopsy needle to be introduced.

The needle has a cavity for the biopsy tissue and one cavity to transport the wound plug. A large variety of plugs may be accommodated and inserted into position, as needed by the physician. The biopsy needle also performs spectral analysis until the sample is taken. When the sample is taken the plug is delivered in order to replace the removed sample volume.

The biopsy sample is gently withdrawn applying a little flow of gas to create a positive pressure behind the needle to prevent the compressed tissue from bleeding or leaking fluid. The gas may be an inert gas, as He, of heavier gases, but has to be gently extracted when the plugging needle is introduced.

The plugging needle is introduced and the entire wound is gently plugged mainly at the tissue interfaces with resorbable materials impregnated with the appropriate drugs. The goal of all this technology is to inflict minimum damage, and prepare the patient for immediate discharge to home and a fast recovery, with minimal risk of infection. If drug implantation is done, this begins immediate treatment of his disease.

The proposed devices to apply this novel method is a family of needles with various diameters starting from 0.5 mm up to 6 mm, depending on the purpose. The narrow gauges have only 3 capillary tubes inside. One to apply the laser excitation pulse and one to get the response from the cells excited in front of the needle. These needles have a hydrophobic deposition on the tip to prevent liquids penetrating and may use a gas bubble in order to have good transmission of the optical signal. The third one is used for liquid transfer purposes. Once the needle is in the tissue, it may withdraw liquid in order to perform a other pathologic analysis or culture, may introduce some gases, or inject liquids, with drugs or sealants to seal the wound and the extraction pathway.

If the spectrometric data obtained is not enough to establish a diagnosis, a foil for sheathing is introduced guided by the needle until reaching the target organ. A more complicated needle with higher diameter is introduced having better directional spectrum collection capabilities and more measurement instrumentation on its tip, increasing the resolution and localization of the data about the tissue. The passage from a narrow needle to a higher diameter is made using the sheath's expandable properties.

If the decision to perform a biopsy sampling was taken, the 3 to 6 mm diameter needle is used, enlarging the sheath's diameter. The 3 mm biopsy needle may be the conventional type used for biopsy modified with a plug in the tip, while the larger diameters will have a cavity to carry the plug and means to deliver it at the place of the biopsy cut. In order to improve the size of the sample, a vacuum may be applied pulling the tissue into the cutting apparatus. The needle also brings in a plug that may be solid or gel.

The plug may be a resorbable material, or a micro-implant with sensors, or radioactive material, or drugs. It may be elastic, transported compressed and when released it expands to fill the biopsy hole, later being absorbed by the body. The opposite side of the cutting blade that pushes it into the tissue makes the plug placement. A liquid or gas may be pumped in to help release the plug into the tissue. The needle is gently extracted simultaneously with adding a pressurized gas or liquid through the capillary tube to prevent any bleeding or fluid leaks from the organ. The sheath remains in its position, and the needle with the biopsy sample is taken out.

Before the sheath is removed, we apply the plugging procedure in order to assure that all the penetrated organs do not leak and will heal quickly. The plugging needle is introduced up into the site that had the biopsy taken, plugs the hole, interface, and the needle is withdrawn., If other tissue interfaces have a threat of bleeding they also could be plugged as the sheathing is withdrawn. Some antiseptic liquid could be injected at different tissue interfaces if desired.

### 2. Best Mode of the Invention

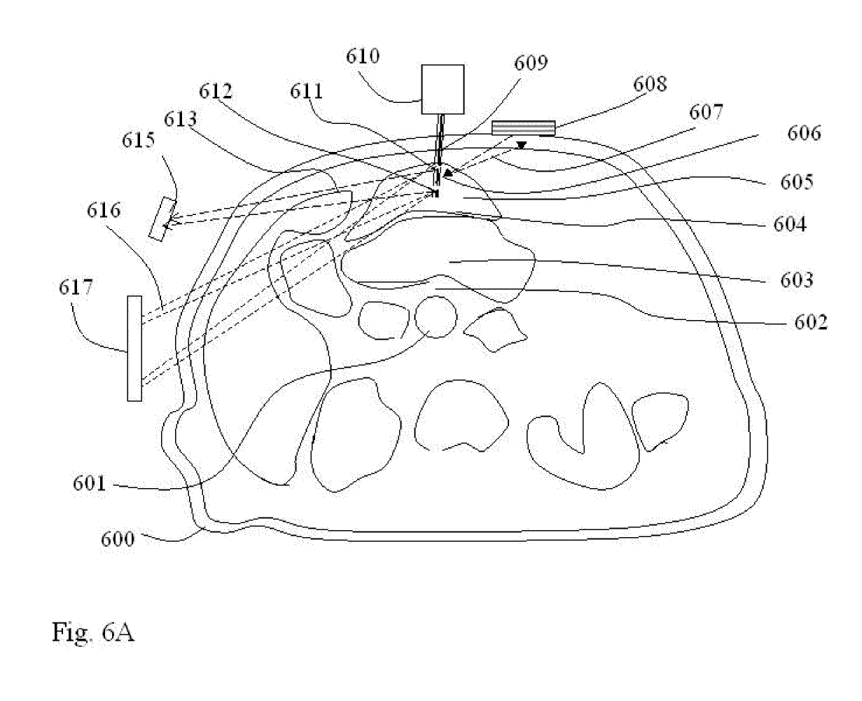

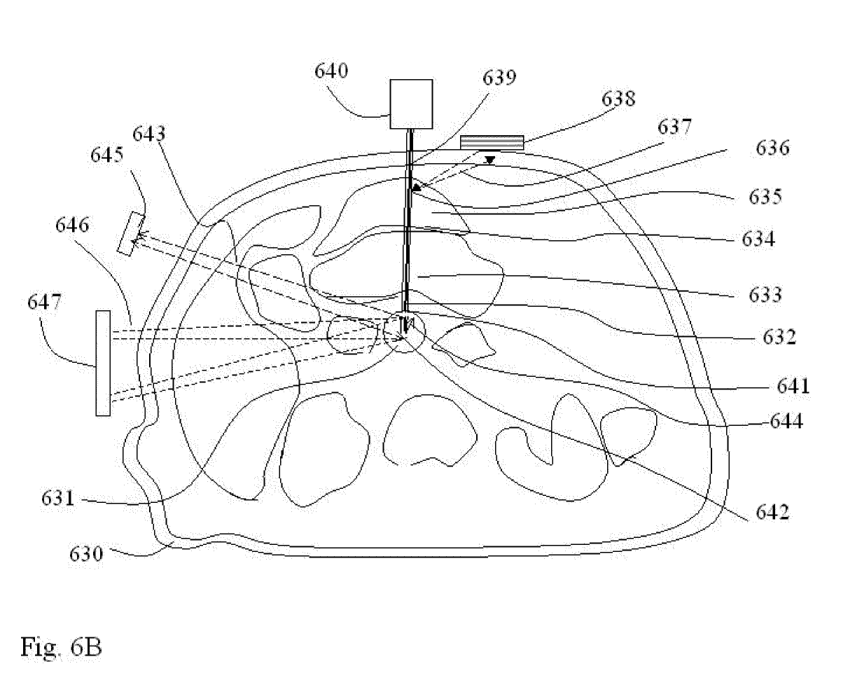

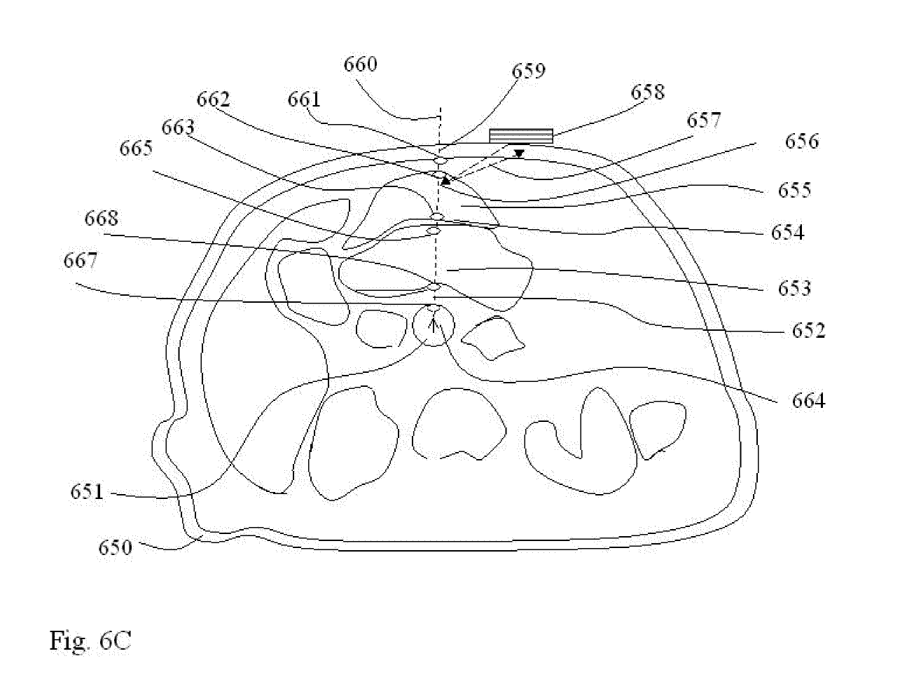

FIG. 6 shows the best mode contemplated by the inventors of a biopsy needle system that has improved features. All the processes are controlled in their spacial positioning by one or another imaging method: CT, MRI or PET machine, or ultrasound phased array imaging, and/or radioactive positioning goniometry, in order to keep the coordinates of the tip of the needle under full control. After biopsy sampling until the tissue interface plugging is done the ultrasound level have to be reduced in order to reduce liquid bleeding stimulated by US pressure. The system have to be used to apply the first medication into the tissue due to the fact that the spectrometric diagnosis is real time, and waiting for corrections to be done after the biopsy complex analysis is done.

The application of a control box over the needle system allows fast accurate aiming into the desired tissue site, safe switching of the needles under positive pressure of controlled atmosphere to prevent leakage. This control box and pressure accessories would be added as customizations to the equipment for specific uses.

### 3. How to Make the Invention

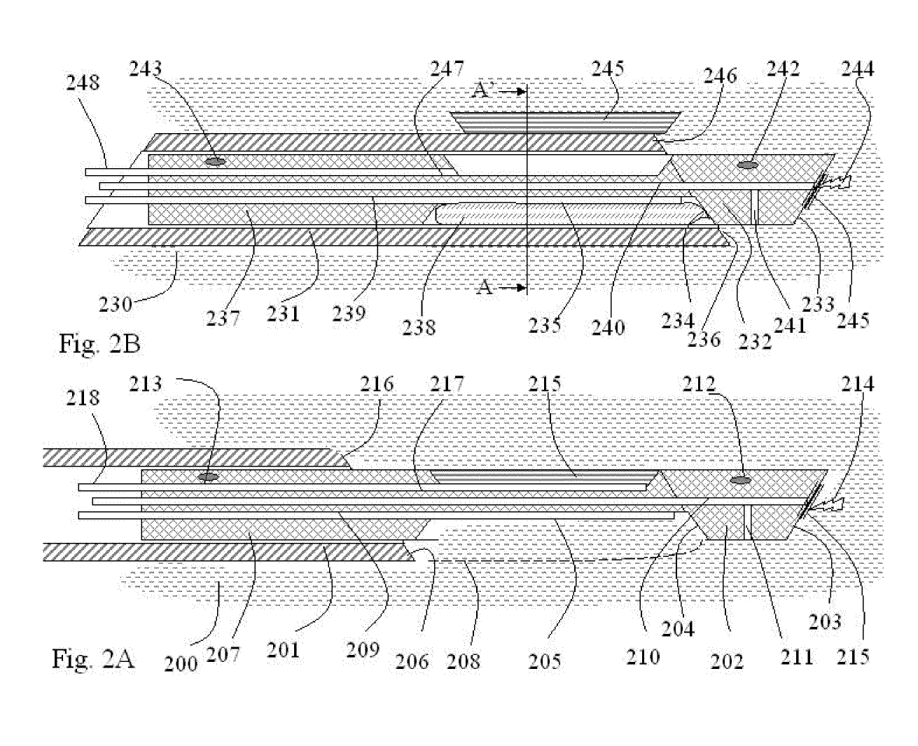

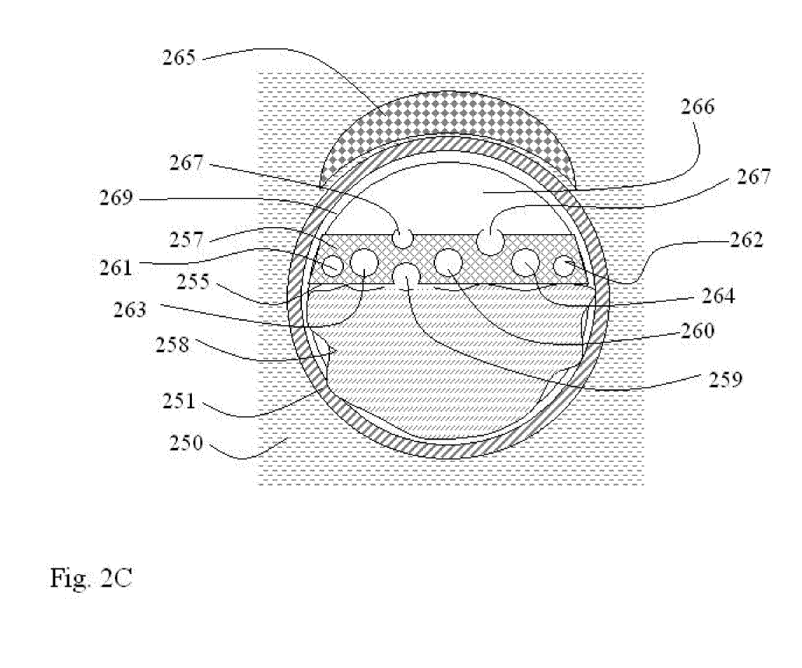

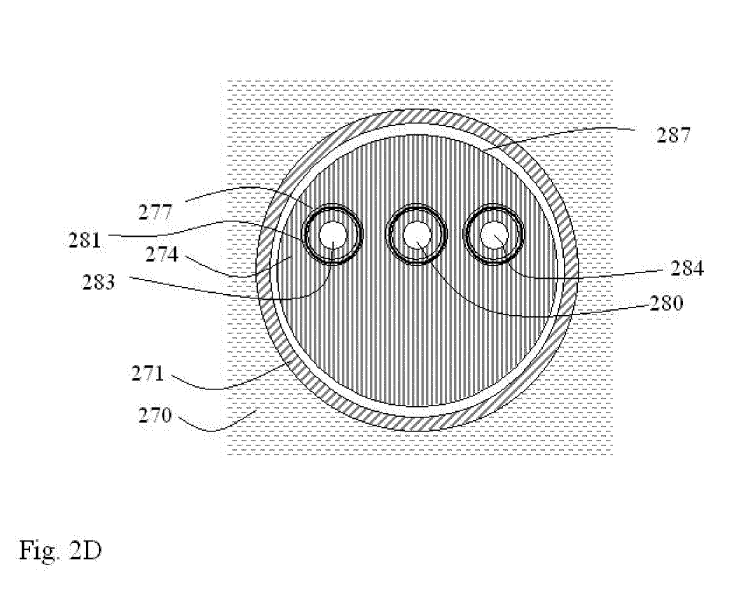

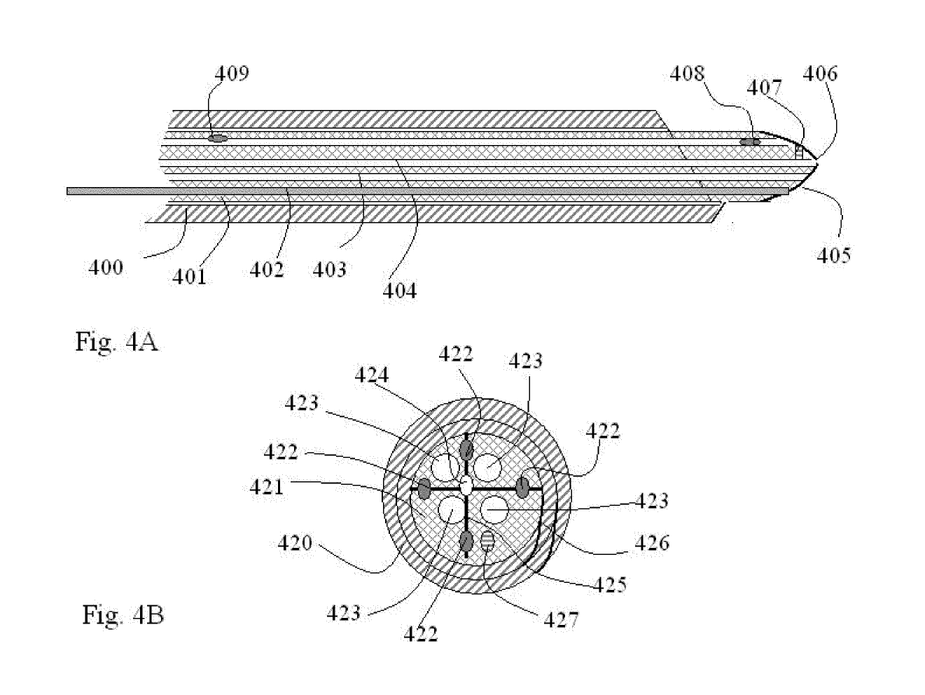

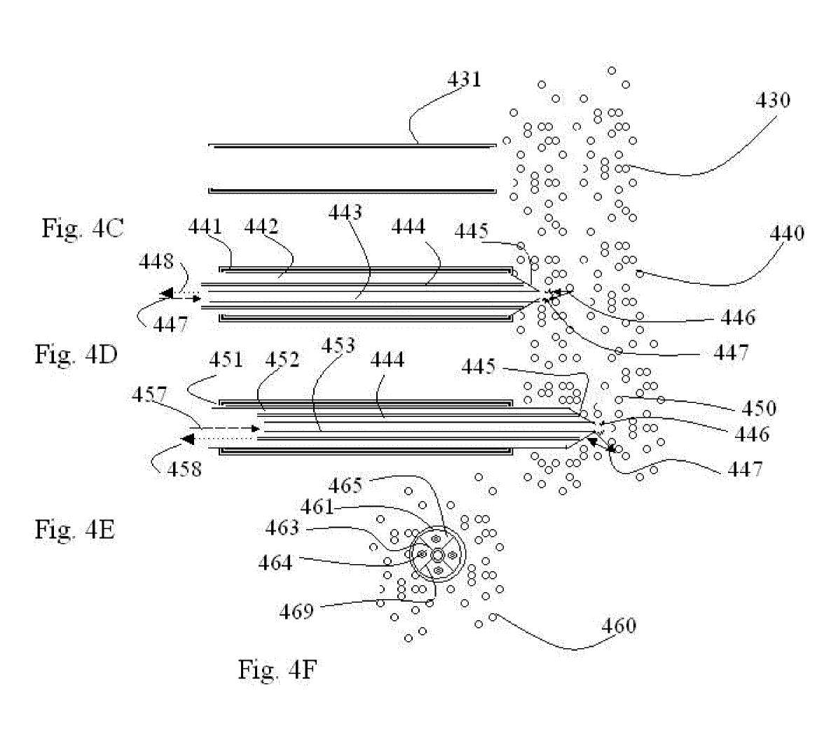

As can be seen from the drawings the needle is the first improvement to be described. The method starts with a smaller steel needle, containing capillary tubing as in FIG. 4, with a partially sharp tip to perforate the tissue. FIG. 2 shows the fabrication of more complicated needles. The actual tissue plugs have to be customized to fit in the biopsy needle's plug deck according to the required dimensions.

Shape retaining resorbable polymers may also be used, so that they open at a certain triggering temperature, warmed by the body heat, then dissolving and being cleared by the blood.

A control box will be attached to each needle and sheath making it possible to control all their functions of positioning, cutting the biopsy, and making measurements with the instruments.

There will be several types of needle developed in order to meet the needs of various applications. But needles and the command and connector boxes will be designed for modularity and interchangeability for cost efficiency. The fabrication will be made using stainless steel and other medical alloys, using electron-welding procedures in order to insert the capillary tubes, and hydrophobic coating for steel sheathing. The plugs will vary for various applications.

### DETAILED DESCRIPTION

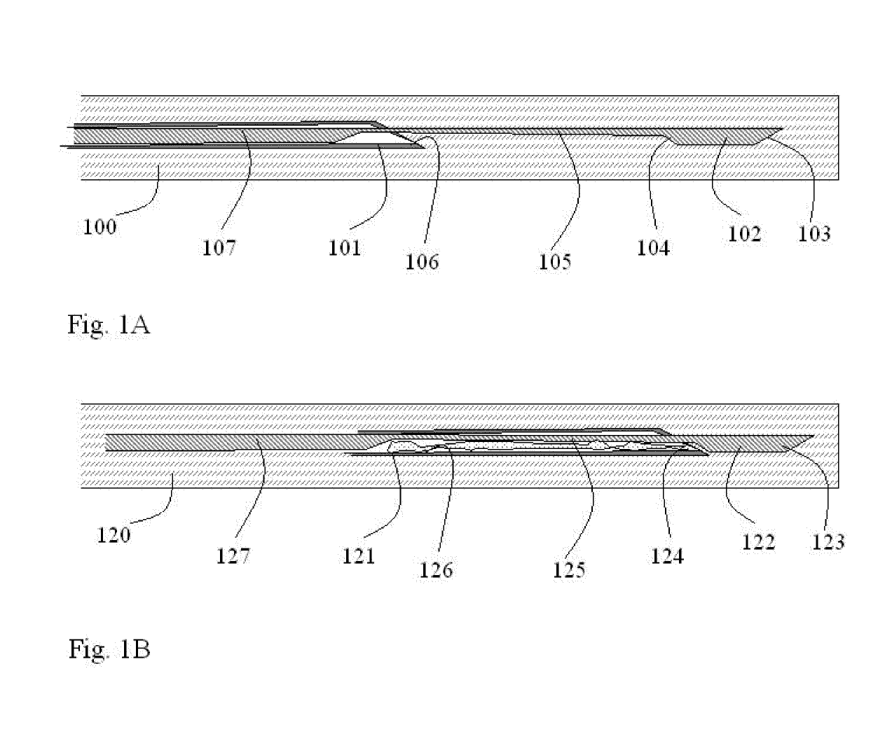

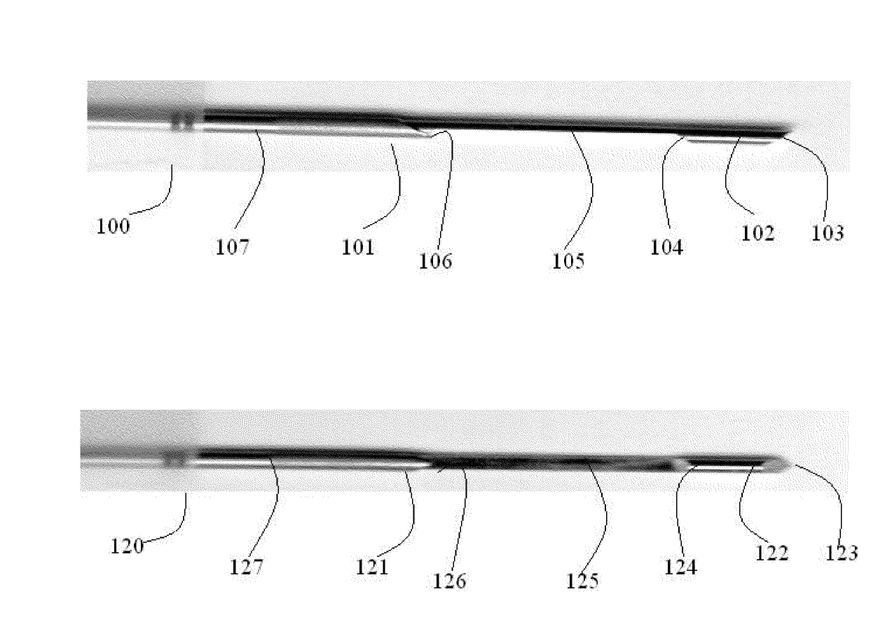

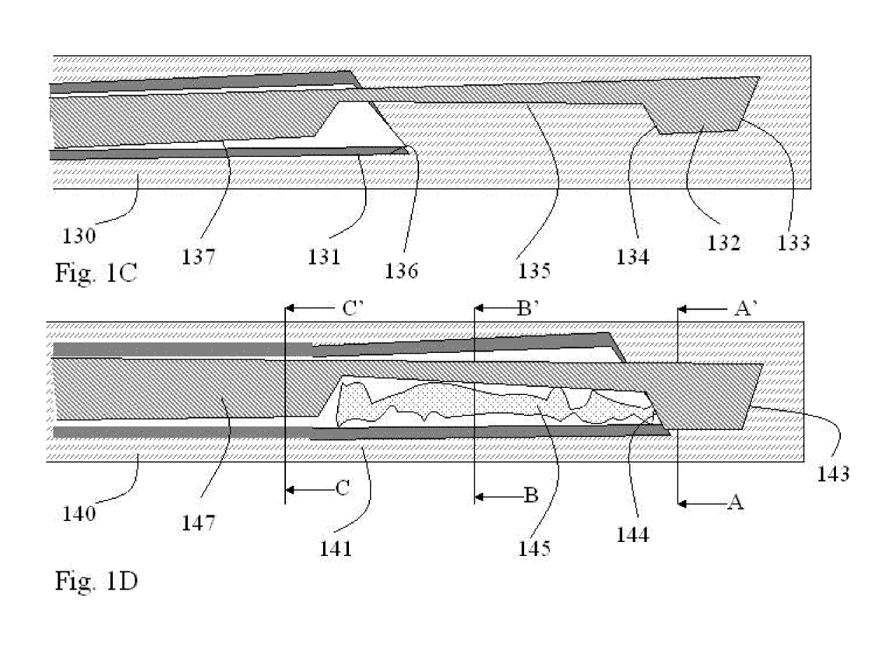

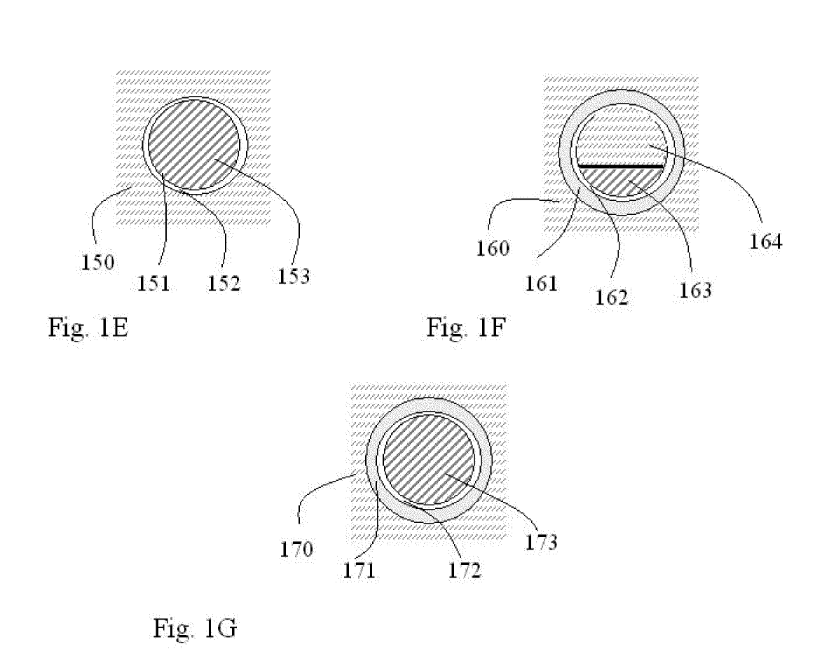

FIG. 1A—shows a present biopsy needle open for sampling, in longitudinal section inserted in the tissue of interest 100. The sampler comprises an external tube 101 that has an echogenic coating making it visible by ultrasound, and a cutting edge 106, which is used to cut the tissue sample. It has an internal rod 107 that fits and slides inside the external tube 101, which has a 19 mm sample notch 105 to ensure sufficient tissue for clinical diagnosis. The notch is terminated by the end-profiled surfaces 104 that is used by the external tube's 101 cutting edge 106 to cut and remove the tissue sample. The front end of the central rod has 22 mm “throw” advancement, cutting the tissue by the edge 103. The front tip body 102 is used for tissue sample forming.

The operation is in 3 simple steps, as locked in launching position, 22 mm behind the tissue of interest, when the launch button is pressed and the central rod is forced quickly forward, triggering the launch of the external jacket cylinder that follows and cuts the sample and insulates on the notch. In various circumstances it is possible to trigger the cylinder-cutting jacket so that the sample is smaller. The profile shows also that in hydrodynamic viscous condition the notch is bending outwards making the rigid jacket pull it back and stress the sampled organ.

FIG. 1B—is showing a present sampler needle closed after sampling, longitudinal section inserted in the sampled tissue 120. The central rod 127 is in the maximum elongated position of about 22 mm it reached after being forced in. After this, springs push the external cylinder tube 121 to slice the tissue 126 until reaching the terminal facet 124, sealing the tissue on the notch pad 125. In this position the end of the central rod 122 and its cutting facet 123 is inactive. The sampling process is finished and the whole assembly is withdrawn from the body leaving a serious penetration wound behind. The actual technique uses compression to limit the bleeding that is not fully successful for tissues placed deep in the body.

FIG. 1C—shows present biopsy needle open for sampling longitudinal section with diameter zoomed by a factor of 4. The sampler is inside the tissue of interest, 130, after the penetration is made. The internal rod 137 has been launched and its tip 133 cut and penetrated the tissue up to the maximum elongation position. The tip body 132 is giving support for the cutting edge 136 of the outer needle tube 131 to close on its tilted wall 134 and lock the tissue in the notch 135.

FIG. 1D—shows present biopsy needle closed sampling longitudinal section with diameter zoomed by a factor of 4, with the aim of better showing the process details. The central rod 147, maintained its position in the tissue with its head 143 passing the area of interest delimited by the cutting tilted pad 144 by a few mm. The external cylinder 141 having sharp edges has been advanced and cut the tissue 145 seizing it inside the notch. From the technical point of view there are several cross sections of interests AA′, BB′, CC′ that will be detailed.

FIG. 1E—shows cross section through AA′ plane in FIG. 1D in the head of the central rod as is immersed in the tissue 150. The outer tube 131 is seen in back as a contour line mainly touching the surface of the head 132 coated in ultrasound reflective material, and having a solid bulk 153.

FIG. 1F—shows cross section through BB′ plane in FIG. 1D in the middle of the notch. The needle is immersed in the tissue 160, having the external cylinder 161 over the notch pad 163 and containing in the “cargo” space the sampled tissue 164, while on the opposite side of the notch bay rod 163 is a gap 162, that allows smooth movement between the parts.

FIG. 1G—shows cross section through CC′ plane in FIG. 1D immersed in the tissue 170. The outer cylinder 171 covers the inner rod 173 leaving a small gap 172.

FIG. 2A—shows the new improved sampler/biopsy-gun according to an embodiment of the present invention in the tissue in open position, ready to cut, immersed in the tissue of interest 200, in the final position after the “throw” phase was accomplished and the inner piston 201 has fully advanced in the tissue, just before the external cylinder 207 advance is triggered.

The central rod according to the present invention, maintains the sampled tissue notch (bay) 205 almost identical with the previous versions, with the difference that its size may be varied according to the needs, but on the opposite side is a similar size notch that carries a plug impregnated in drugs that is meant to replace the missing volume, stop blood leaks and apply a treatment to the diseased organ. It also prevents the fluids released by the biopsy cutting to spread into other tissues.

This is a better solution than applying pressure, and minimizes the risk of spreading of disease among good cells.

The central rod 207 holds the sampler notch 205, delimited by the facet 204 that is the end of the cutting range of the external cylinder 201, that has a sharp edge 206 that may follow the cutting path 208 so that the sampled tissue gets compressed in the notch 205, or it may only cut without compression if the cutting edge is near the internal surface. In order to better hold the tissue in the sampler notch a slight vacuum is applied, that makes the tissue follow the notch 205 profile.

The head of the central rod 202 is not a bulky metal piece with a cutting edge 203 only.

The invention is proposing a head with a complex structure to take measurements from the internal tissues directly.
It contains a set of 2-3 wave-guide hollow tubes 210, used for spectroscopic measurements. A UV-Vis laser pulse 214 is guided in the front of the cutting tip during its advancement that produces the atomic and molecular excitation of the tissue. A Vis-IR spectrometer is analyzing the radiation collected by the lateral tubes presenting in real time the molecular content of the penetrated tissues. To keep the tubes open they may be protected by a hydrophobic material 215, that repels liquids or gas pressure may be used.

In order to get a good positioning accuracy two x-ray sources 212, 213 are placed in the rod using low radiation energy as 59 keV ²⁴¹Am, 122 keV ⁵⁷Fe, 392 keV ⁹⁰Ir or 661 keV ¹³⁵Cs that may be used for accurate coordinate positioning and for stereoscopic radiography of the tissues.

The central rod head also contains a tube 211 that may be used to inject various drugs inside the tissue. These liquids may carry drugs or other liquid or dispersed solid materials to the diseased organs being biopsied.

The plug notch, 215 contains the plug that may be solid or flexible, that is pushed into position by the profiled edge of the outer cylinder 216, possibly aided by liquid or gas pressure transmitted through the capillary tube 217. The internal rod carrying the capillary tubes 218 is modular and may be simply replaced with another module using a modular coupling structure.

FIG. 2B—shows the new sampler immersed in tissue 230 in closed position after cutting and sampling the tissue 238. The outer cylinder 231 was advanced, which cut and sealed the tissue 238 in the notch cavity 235, also stabilized by a slight vacuum coming through the capillary tube 239. The cutting blade 236 in close contact with the surface 234 seals the sampled tissue cavity 235. The sampler head 232 could release some drug as a fluid through the nozzle 241 for various therapeutic purposes. The tip surface 233 with hydrophobic coating 245 is in the same position, and the laser 244 may still be firing in order to obtain high resolution Vis-IR spectral analysis and detect the drug interaction with the tissue.

Another main embodiment of the present invention refers to the fact that the plug 245 is now released at the device end 231 by a combined action of the pressure applied through the capillary tube 247 and the advancing cutting blade 246 that pushes the plug into the tissue to reduce its bleeding.

This plug is made of tissue specific absorbing material, drug impregnated, giving it both mechanical and chemical action. After the device withdraws it will expand inside the space left by the sample removal as sets of conic shaped sub-plugs separating the wound space into sub-volumes and preventing leakage or spread of disease. The plug structure is impregnated with various chemicals, to promote healing of the tissue and may provide an early treatment of the suspected tumor or other disease.

The radioactive sources 242, 243 added on the inner core 237 are used to confirm the positioning of the sampling volume in body coordinates, and in the right organ or tumor location, since we know that the inner organs are constantly changing their position. The stereoscopic X-ray imaging and syringe vector coordinates determination information corroborated with the ultrasound, or other positioning systems like CT, MRI in real time certifies the quality of the intervention and minimizes the collateral damage probability Assuring proper positioning of the biopsy sample might use stereoscopic X-ray, ultrasound, or other systems like CT, MRI. The chemical map along the entry pathway, gives supplementary information and real time diagnosis, in some cases eliminating the need for tissue sampling, in which case the device diameter could be under 1 mm.

The capability of injecting liquids through the nozzle 241 opens the possibility of using UV-polymerizing plugs activated by the laser at the device end before its removal from the tissue. The system may be a multi-gun device, starting with a laser/spectroscopic needle. Then using the same pathway if the Vis-IR molecular spectroscopy information is not conclusive, a tissue sampling needle is added for the appropriate sample dimensions and the plugs drugs customized based on molecular spectroscopy information. After the sample is extracted the biopsy needle is taken-out and replaced with customized tissue plugging device prior to removing the outer tubing The modular coupling 248 helps in dynamically interchanging the device cores.

FIG. 2C—shows cross section AA′ in FIG. 2B, showing the plug 265 outside the insertion tubing 251 embedded into the tissue of interest 250.

This represents a main embodiment of the present invention, which not only samples the tissue for rapid analysis, but this rapid diagnosis may allow local treatment of the disease to be implanted into the organ at the same treatment session. Because this invention causes minimal tissue damage, healing of the biopsied organ is promoted; and the possibility of beginning immediate treatment adds to its benefit compared to previous techniques.

The figure shows the section through the sample retaining notch and finds the sampling assembly 251 in closed position with the sample contained in the notch 255 following the desired cutting line 258 and stabilized inside by vacuum applied through the capillary tubes 259. A special hydrophobic inner coating 269 keeps any fluids in the area from spreading inside making a seal between the inner core 257 and the tubing wall 251.

The inner core is formed to create the two bays for sample notch 255 and for plug bay 266. The rectangular tray 257 contains inside a set of capillary tubes used for delivering drugs and other liquids. 261, 262. Gases may be delivered as well with the necessary precautions required for safety.

The central tube 260 is used as guide for transmitting pulsed signals as IR, Visible or UV laser and may have a hollow core with a transparent window at the end or a hollow covering. The central core conducts optical signal pulses that illuminate the tissue, which variously reflects the pulses according to specific spectral absorption properties in various healthy or diseased tissue types. The reflected optical signals return through the tubing core wide band transmission of the micro-tube 263 which then 264 passes the signals to a spectrometer that analyses them making the molecular recognition. The tubing wall 264 may also carry electric wires to measure other properties of the tissues encountered.

The plug bay 266 has a special design to hold a plug 265 and release it after the sampling into the tissue gap created by the biopsy. The plug notch contains a set of capillary tubes that may be used to apply a liquid or gas pressure to release the plug from its support and place it in a position that the outer tubing 251 can properly place it in the tissue 250. Thus when the outer tubing withdraws, the plug remains in the gap made by the biopsy and elastically expands plugging the gap.

The plug material is designed to be absorbed by the tissue over time. It may be impregnated with a wide range of chemicals with various functions.

FIG. 2D—shows the front view of the front-end of the sampling device 277, with the head cutting surface 274 having the cutting edge 287. The surface is coated with a hydrophobic material repelling water 277 mainly around the exit of the capillary holes, and may hold transmission windows 281.

The central hole 280 is used for the exit of optical pulses as from a UV laser, to illuminate the tissue; the one or two lateral channels 283 and 284, then collect the reflected and emitted light. By turning the device on its axis a circular profile of the surrounding area may be obtained, and tissue fiber may be identified, but in this case the cutting edge device's profile 287 is inappropriate for this operation.

One of the capillary passages could be used for a micro-tip sensor to measure the electric potential or other properties of the tissue.

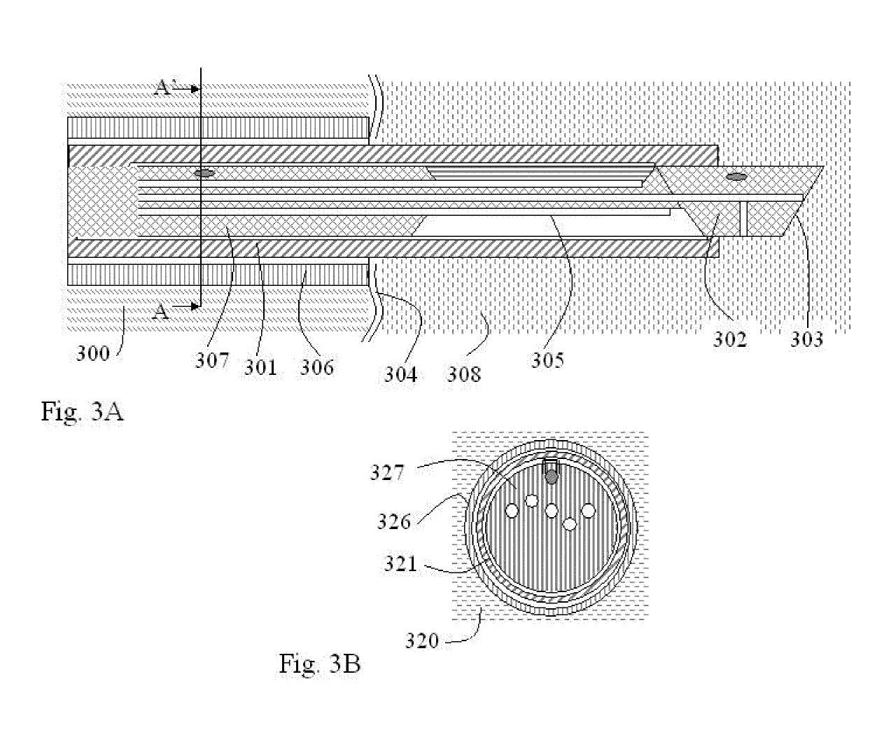

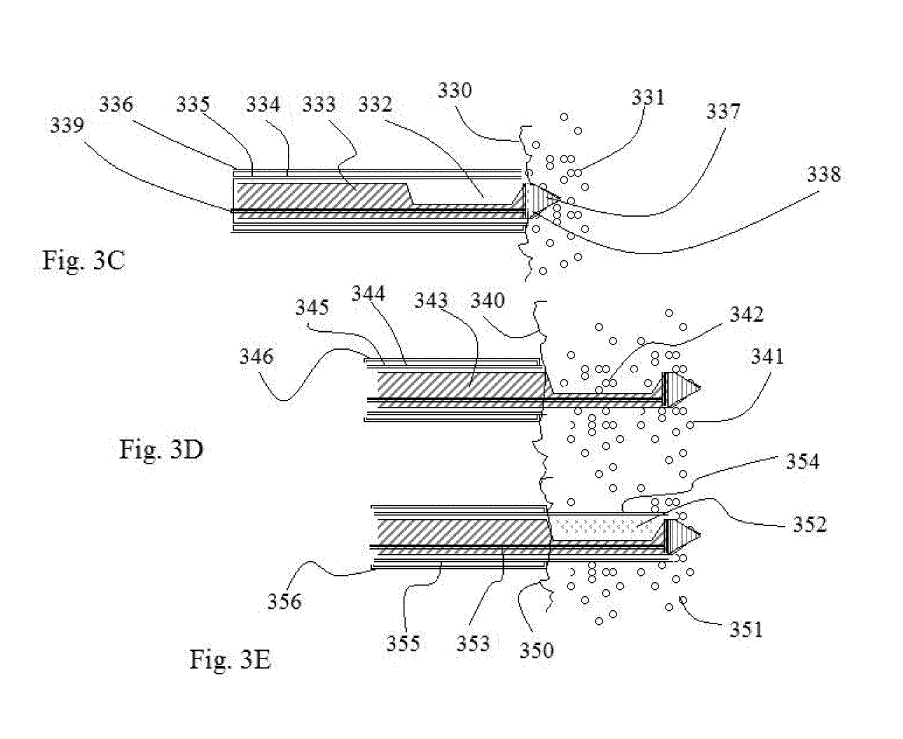

FIG. 3A—shows the tubing array with supplementary outer shell for protection of the other organs penetrated, in order to prevent blood or fluids spreading to them after the device is withdrawn. The sampling gun is withdrawing in a protection tube that remains in the body, which is then cleaned by introduction of a specialized gun, then a plugging gun is introduced and the tissue gap is plugged as the outer tubing shell withdraws.

The biopsy sampling is supposed to come from the diseased tissue 308, but in order to get there it has to penetrate the skin 304 and any interposed tissue 300. It is desired that no diseased material 308 spread into the healthy tissue 300. For this purpose a protective tube 306 will be added over the outer tubing 301, that will stop in the interface 304 between the healthy 300 and sick tissues 308. The tubing 301 will be further introduced until the inner rod 307 is in the tissue of interest 308 with the sampling notch 305 fully immersed and the head 302 slightly passing through but preventing the front cutting edge 303 to penetrate.

In this condition the sampling is performed and special care is applied to decontaminate and plug the interface 304 then decontaminate again and withdraw the protective shell 306 and treat the wound.

FIG. 3B—shows cross section of the sampling needle in AA′ position in FIG. 3A. The protective shell 326 is separating the healthy tissue 320 from the contamination with sick tissue by the withdrawing tubing body 321 containing the sampling head 327.

FIG. 3C—Shows the biopsy gun obtaining tissue 331 for pathology, with other sheath 336 over biopsy gun 333 in position before penetrating the tissue 331. The needle is placed on the surface of the tissue of interest 330, with the outer sheath 336 outside the tissue interface, but close to it. The sampling gun 333 has a sample volume 332 and an outer cutting cylindrical sheath 334 separated by a space 335 from the outer protective sheath 336. A removable tip 337 that can be separated from the needle 333 any time along the interface 338, using a pressurized liquid inserted in the capillary tube 338 was added as a potential improvement to a previous patent U.S. Pat. No. 5,080,655, where a resorbable tip impregnated with drugs may be left in the wound, possibly in 2 stages: one in the far end of the sampling wound triggered by the sheath movement and one at the interface with the tissue, triggered by liquid pressure or wire actuator using the tube 338, bordering the sampling place at the both ends, and leaving medicine fulfill the space.

FIG. 3D—Shows the biopsy gun obtaining tissue for pathology, with other sheath 346 over biopsy gun with biopsy gun 343 extended in the tissue 341 of the targeted organ. The needle 343 is penetrating the interface with the tissue of interest 340, with the outer sheath 346 left outside the tissue interface 340, but close to it. The sampling gun 343 has a sample space 342 loaded with tissue and the outer cutting cylindrical sheath 344 separated by a space 345 from the outer protective sheath 346 is still withdrawn in the protective sheath waiting the launch command to be activated.

FIG. 3E—Shows the biopsy gun obtaining tissue for pathology, with other sheath 356 over biopsy gun 353 with the tissue specimen 352 loaded. The needle that collected tissue of interest 350, with the outer sheath 356 outside the tissue interface 350, but close to it The sampling gun 353 has a sample space 352 loaded and the sample was cut by the cutting cylindrical sheath 354 that is separated by a space 355 from the outer protective sheath 356 to equalize the pressure inside the protective sheet during the withdrawing of the biopsy gun. This prevents the creation of vacuum behind the biopsy gun assembly that would pull liquids and matter from the targeted organ tissue.

FIG. 4A—shows another embodiment of the present invention in a longitudinal section through the needle with profiled cutting head. The idea behind this development is to cause the patient the minimum harm by minimizing the invasive device's size and impact. The biopsy procedure using this method would be a multiple stage process.

First a very small diameter needle in the range of 0.5-1.5 mm is introduced coated in a foil twisted over several times and forming the previous cylindrical shell.
The idea is to use laser illumination and UV-Vis-IR spectroscopy to analyze the tissue with minimal cutting to penetrate into the tissue. The laser will illuminate while the head will rotate and detect fiber direction and blood vessels. With this first stage device, tissue sampling could be made by activating the cutting blades through a sonic system that make it vibrate at the tip, simultaneously releasing some anesthetic or antibiotic.
The returning optical signals will be analyzed by a spectrophotometer. If the spectral analysis is good enough for diagnosis, to complete the procedure the plugging tip will be inserted for sealing the holes. But if still more tissue is needed for diagnosis, the next stage biopsy needle will be inserted making the rolled foil expand to better seal the hole.

The penetrating device 401 is coated by a rolled foil 400 internally and externally with hydrophobic surfaces that prevent liquids intrusion. The penetration core contains inside several capillary tubes 402, 403, 404, all along its length, performing multiple functions.

The device has a sharp tip with good optical properties 406 connected to the central channel 404 where a laser beam is used both to illuminate the tissue and to excite molecules for spectrometric analysis. The tip profile is optimized to easily penetrate the tissue with minimal damage and has a set of micro-blades that can be vibrated using sonic power in order to make the desired cut in the tissue. Through an orifice 407 placed on the tip an anesthetic is released making the penetration painless, or a drug can be injected in order to make the operation antiseptic.
For guiding and imaging purposes the penetration rod has two point radioactive sources 408 and 409 allowing a precise positioning inside the body.

FIG. 4B—shows a front view of the sampler with profiled cutting head, another embodiment of the present invention, based on the principle of making as little as possible wound damage and obtaining as much as possible information, with no collateral damage and minimum harmful impact. For this, it is desired to start with a small puncture, advance as gently as possible, under full control. To achieve that we have to start small with the optical probe, and if this is not enough we may apply the larger sampling system, gently enlarging the diameter up to the appropriate size for the second stage device.

The outer sheath 420 is made of a elastic foil rolled several times and having the ability of varying its diameter easily from small to large gauges. It covers the penetrating needle 421 that in this minimal version does not have the plug insert device. The penetrating device surface is accommodated to the hydrodynamics of the penetration, delivering mainly axial drag forces. The central tip, 424 is used for illuminating the path spreading the laser light in the near-by tissue and also for smooth penetration. The penetration is made by the blades set 425 to activate by the pressure driven into a micro-piston bellow like cavity 422 that pushes the blades out and retract inside to allow the rod to spin +/−90 deg. This is to mobilize tissue fragments so that the window 424 visualizes the reflected light signals coming from the cells or tissues, which are sent back through the optical guides 423. If the tip 421 is ceramic the blades 425 may be used for pH measurement while the needle penetrates the tissues, while the optical system performs a spectrometric analysis of the tissue.

FIG. 4C—shows a tissue 430 scanner with the biopsy gun, with empty sheath 431 placed in front of the targeted organ 430.

FIG. 4D—shows a tissue scanner with hollow-core optic fibers 443, 444 smoothly penetrating the targeted tissue 440. In the central hollow-core optical fiber 443 an excitation laser light 447 is applied that is reflecting 446 and is exciting the atomic and molecular optic radiation levels of the targeted tissue 440, and is backscattered and collected back 447 in the central hollow core optic fiber and guided 448 towards a spectrometric analyzer that may detect the presence of various molecules.

The initial reflection 447 may be collected in surrounding optical guides 444, embedded in the needle body 442 with a solid tip 445. The optical tip may be active all along the entry path or only in the targeted tissue. The tip 445 started to penetrate the targeted tissue 440.

FIG. 4E—shows a tissue scanner with hollow core optic fibers 453, 454 smoothly penetrating the targeted tissue 450. In the central hollow-core optical fiber 453 an excitation laser 457 is applied that is reflecting 456 and is exciting the atomic and molecular activity of the targeted tissue 450, and is backscattered and collected 457 in the central hollow core optic fiber and guided 458 towards a spectrometric analyzer for analysis.

The initial reflection 457 may be collected in surrounding optical guides 454, embedded in the needle body 452 with a solid tip 455. The optical tip may be active all along the penetration path or only in the targeted tissue. In the FIG. 4E the tip 455 penetrated the targeted tissue 450 up to the level of sampling. It may penetrate more or stop there.

FIG. 4F—shows the tissue scanner front view of the tip 465 showing the penetrated tissue 460 surrounding the needle in the position shown in FIG. 4D. The outer protective sheath 461 is visible in depth. The tip has a bunch of micro-blades 469 used to gently cut the tissue on forward motion. It has a central hollow core optical guide 463 used for laser excitation of the sample; the reflected fluorescence back-scattered signals are captured and transmitted back through the lateral hollow core optic fibers 464 for spectral analysis., for multi-point molecular imaging, embedded in the needle's body 463.

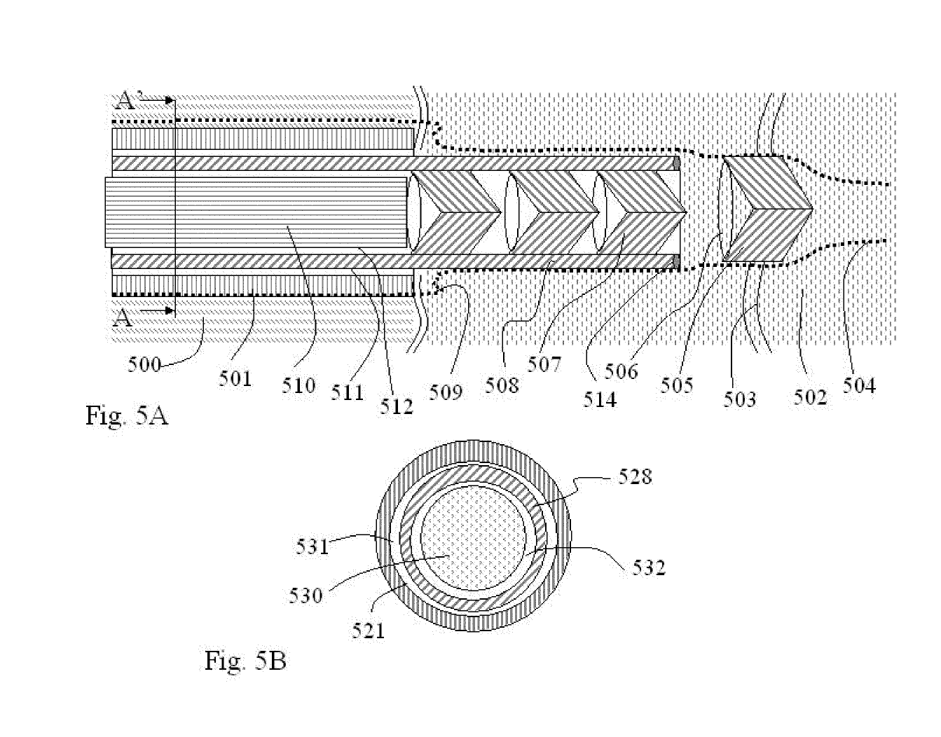

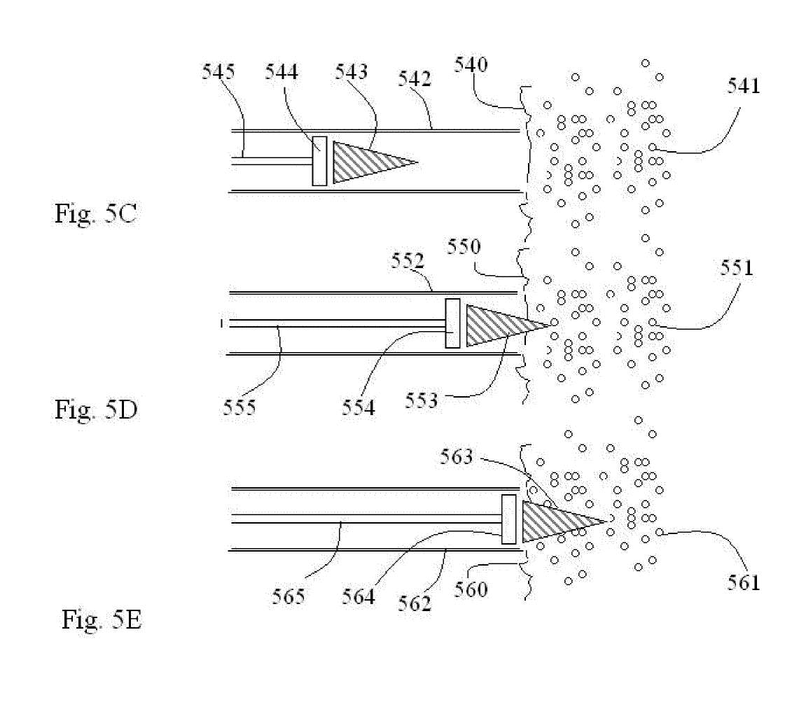

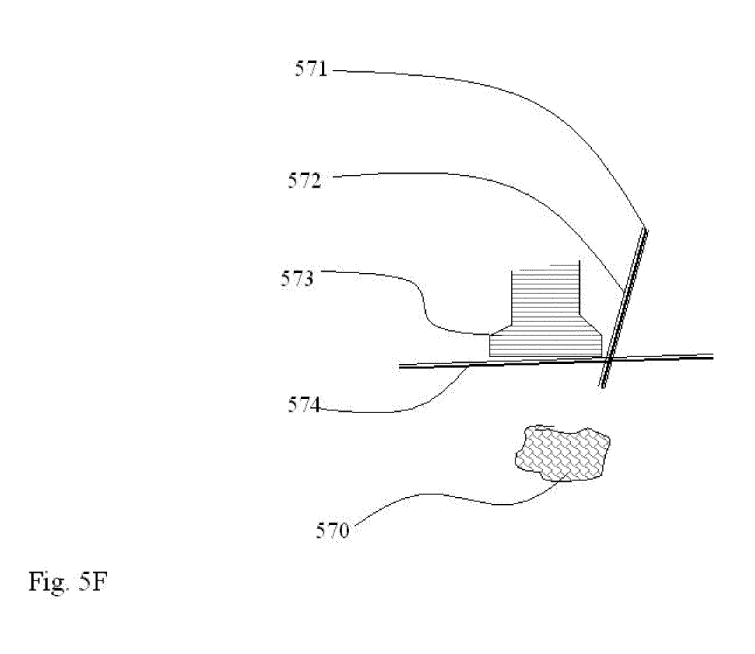

FIG. 5A—shows in longitudinal section of the apparatus with internal plug releaser that comprises an external shell 501 that may be a cylinder or a cylindrically rolled foil acting as a cylinder but having expandable diameter remaining in the wound 509 after the spectrometric diagnosis needle or the sampler have been withdrawn, an inner tube 511 acting as a cylindrical guide that hosts inside a piston 510 that pushes a set of customized plugs 507.

After the tissues of interest 502 have been investigated by remote analytical means (spectrometer, pH-meter, imager, etc.) or by taking a tissue sample, the biopsy gap 504 at the organ 503 is plugged 505 to prevent bleeding or spread of disease. It is supposed that in the case of a tissue sample the sampler released the appropriate plug to compensate for the missing tissue, discussed above, and the present plugging operation is designed to improve the healing of the penetration wound path.

The inner tissue 503, released plug 505, has a conical expandable structure that mechanically seals, and a drug reservoir 506, meant to enhance the healing. The entire structure is absorbed in the tissue later.

In the piston other plugs 507 are inserted and released gradually while the piston 508 is withdrawn from the tissue. When it reaches the limits of a protective tube 501 the piston solidifies with it, acting as a single tube and releases the appropriate plug to seal that interface that separates the tissues 500 from the intermediary tissue 509 in the wound zone. By this procedure releasing a series of plugs, the healing time is reduced and the patient could be safely released to go home. A similar technique may be used to seal military penetrations, but the plug has to be more complex introducing substitute parts for the damaged tissue. In order to prevent the liquids intermixing a hydrophobic coating may be applied 511,512 on the cylinders surfaces. The tube holding the plugs has one or two radioactive sources 514 for localization and imaging purposes.

FIG. 5B—shows in cross section AA′ in FIG. 5A through the piston for organ interface plug releaser piston 530. The system comprises an exterior shell 521, the cylinder 528 holding the piston 530 separated by hydrophobic layers 531, 532 that prevents water and body liquids inter-mixing.

FIG. 5C—shows the longitudinal section for the introduction of hemo-static material using the protective sheath 542 at the introduction. It comprises a piston 544, which may have air ducts in order to allow air to flow avoiding creating pressures inside during compression on input path. It has a rod 545 and a piston disk 544, on which the plug 543 is placed. The external protective sheath 542 is still at the interface with the targeted organ 540 from where the tissue 541 was sampled.

FIG. 5D—shows the longitudinal section for the introduction of hemo-static material using the protective sheath 552, ready to implant in the targeted tissue 551. It comprises a piston 554 which may have air ducts in order to allow air to flow avoiding creating pressures inside during compression on input path. It has a rod 555 and a piston disk 554, on which the plug 553 is placed. The external protective sheath 552 is still at the interface with the targeted organ 550 from where the tissue 551 was sampled.

FIG. 5E—shows the longitudinal section for the introduction of cyto-hemo-static material using the protective sheath 562 implanted in the targeted tissue. It comprises a piston 564 which may have air ducts in order to allow air to flow avoiding creating pressures inside during compression on input path. It has a rod 565 and a piston disk 564, on which the plug 563 is placed. The external protective sheath 562 is still at the interface with the targeted organ 560 from where the tissue 561 was sampled, and the plug is inserted by the advancement of the piston and cyto-hemostatic plug 563 introducer, up to the biopsy hole.

FIG. 5F—shows a section of the utilization of the scanner 571 with the protective sheath 572 and its introduction in the body 574 aiming to the organ of interest 570. It is assisted by a ultrasound-imaging device 573 only representing a simplistic version of application of this novel technology. The protective outer sheath 572 is used for guidance of either scanner or biopsy gun inside 571. If the scanned information is insufficient for diagnosis and a biopsy is required the operator will exchange the scanner with the biopsy gun. After the diagnostic study is complete the operator will place the plug using the introducer that releases the cyto-hemo-static plug into the tissue and remove the protective sheath 572 from the body 574.

FIG. 6A—shows a schematic view of an abdominal biopsy-sampling process. The process is in the first phase when the body 600 is placed on the coordinates control table, in order to investigate a tissue of interest 601.

After study, the operator decided the optimal trajectory that will make the sampler 609 penetrate skin and intermediary organs 603, 605 having the interfaces 602, 604. The needle gun 610 is placed in position and the insertion of the needle is closely monitored by the ultrasound localization system 608 that shoots a beam that hits the echogenic spots on the needle and reflects captures the reflected ultrasound 607 into the phased array receiver. The needles according to present invention may also have two or more radioactive gamma sources 611, 612 emitting different energies in order to be distinctly visible to the positioning detector 615 that receives the straight line gamma signal 613 and determines the position and direction of the needle being possible to use a coordinate system as with the MRI or CT.

The presence of the radioactive sources inside the body may be used to make a stereoscopic imaging of the inner organs, detecting the position of interfaces with high accuracy, using the CT imaging plates 617 that receives the signal 616 and forms distinct images. In this system the advancement of the penetrating needle is made with moderate speed, all operation being fully recorded for quality assurance purposes.

FIG. 6B—shows a schematic view of an abdominal biopsy-sampling needle 639 in the sampling position. The body 630 is placed on the table and the needle gun 640 is in the optimal position, having the needle 639 inserted in the tissue of interest 631, penetrating the intermediary tissues 633,635 and their interfaces 632,634.

The ultrasound imaging array 638 is in the position receiving the reflected signals 637, and showing the position of the device. The two gamma emitting sources 641 and 642 are accurately localized by the gamma detector 646 that receives their signal 643, and in the same time the gamma imaging plate 647 that receives the signal 646 is imaging the internal organs in the vicinity looking for slightest anomalies. In this position the tissue sampler takes the sample and releases the plug 644 in the same position to compensate for the missing tissue.

FIG. 6C—shows schematic view of an abdominal biopsy-sampling after the device was extracted and the wound plugged. The body 600 is on the table, and inside the sampled tissue 651 remains the plug 664. The boundary of the tissue 651 has the plug 667 inserted, stopping any effluents from leaking into the interface 652, followed by the plug 668 that insulates the tissue 653 in the lower side and plug 665 in the upper side towards the interface 654. The next tissue 655 has also plugs 663 and 662, and the final skin plug 661. The ultrasonic imaging system 658 makes a final check of the operation and the body is ready for recovery with minimal distress produced inside. In the designed time the plugs are fully absorbed in the tissue and no trace remains.

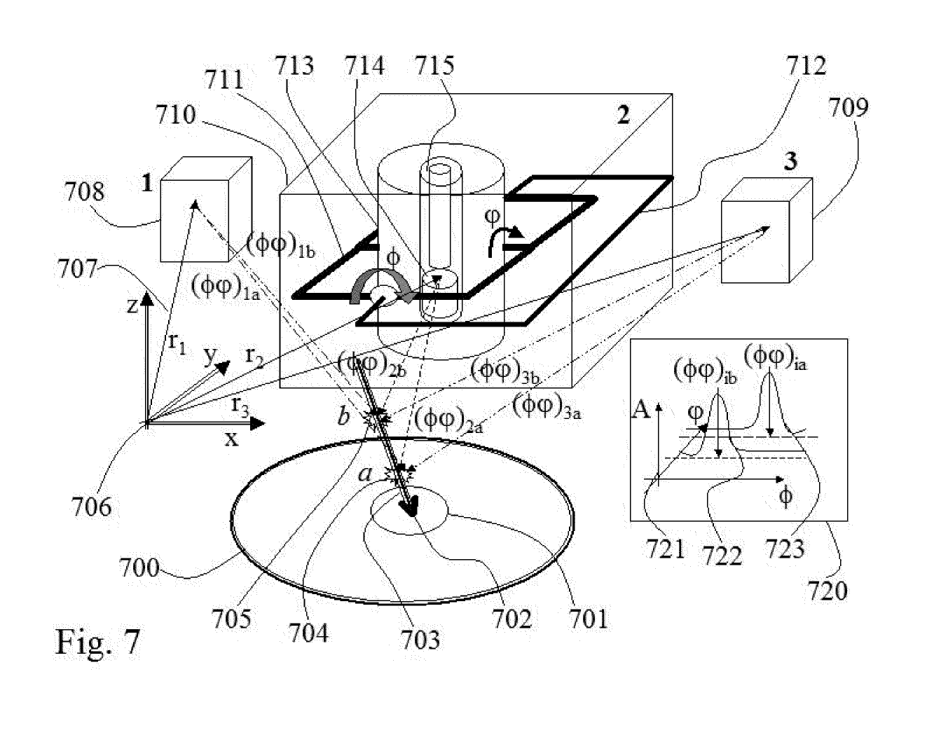

FIG. 7 shows a schematic view of an abdominal biopsy using radio-goniometry made of a 3 goniometric units for triangulation purposes, delivering a localization resolution of about ½ mm inside the patient, with no path deformation produced by reflection or refraction inside the patient's body. The system uses tiny radioactive sources emitting radiation over 500 keV, in order to have very small absorption into the patient's body, typically halving lengths over 1 inch, and having a total radioactivity of several micro-Curie. The total absorbed dose by the patient from this procedure is less than what it absorbs from the natural environment being in range of few micro-Rad. The system presented in FIG. 7 shows a functional diagram of such device. The patient 700 lies on the operation deck and has the organ of interest 701 localized by imaging methods as radiography (RG), ultrasound (US), computed tomography (CT), nuclear magnetic resonance (MRI), or positron emission tomography (PET), having a system of coordinates compatible with the visualization method used.

The optimal access path to the organ 701 was calculated previously and resulted in a set of angles (α, β) applied in the incidence point (xᵢ,yᵢ,zᵢ,) on the patient surface with terminus biopsy point (x_{b},y_{b},z_{b}) 702 where the resorbable needle tip may be lost.

The angles show the advancement direction of the biopsy needle 703, but in order to assure the quality of execution two radioactive micro-sources say containing ⁶⁰Co and ⁵⁶Fe, or ¹³⁵Cs, etc. are embedded in the needle in the points a and b, that have to be found along the line of penetration. On the patient's body may also be added other radioactive micro sources for coordinate localization, knowing that human body is a deformable structure and internal organs may be subject of displacements during operation.

The purpose is to continuously know the position of a and b sources in relation with the body and assure that they stay on the designed penetration path. The optimal path means that the minimum collateral damage is inflicted; blood vessels are avoided, although with the plugs capability, even a penetrated arterial blood vessel may be plugged successfully.

The needle's radioactive tracking system has 3 tracking units, as by chance one is shadowed the other two to provide enough information.

The origin for the coordinate system 706 is somewhere in the operation room, and the scanning cubes 1, 708, 2, 710 and 3, 709, are supported in 3 locations having the coordinates given by the vectors r₁, r₂, r₃, where the center of the detector is placed. The scanning cube is detailed inside having a tungsten collimator shield 713, that holds in its center of mass the radiation detector 714, that may be a GM or a NaI or a CdTe or even better as intrinsic Ge or Si, that has a collimated view outside 715. In order to find the coordinates of the radioactive sources the collimator is moving around the axes x using the actuators placed on the support 711 generating an angle φ and around axis y using the support and actuator 712, generating an angle Φ. The detectors collect a data array looking like 720 where from time to time when they pass over the radiation source they make a peak in counting on the spectral line belonging to that isotope. The information recorded represents the energy channel count value A (as amplitude) as a function of detection angles (φ,Φ) represented in 3 D chart 721 where for example the first peak 722 corresponds to point b 705, while the second peak 723 corresponds to the source a 704.

Calculating the angles of these peaks makes it possible to calculate the directions in space starting from each goniometry point towards the radioactive sources. The volume where the distances from these lines in space is minimum is the likelihood voxel of the radioactive source, and its center may be calculated, giving a resolution under ½ mm or 20 mils. With these coordinates now we may calculate the direction a-b (704-705) and the position of the tip 702 with respect to the body. In order to have an accurate positioning and fast response time enhanced movement algorithms may be used.

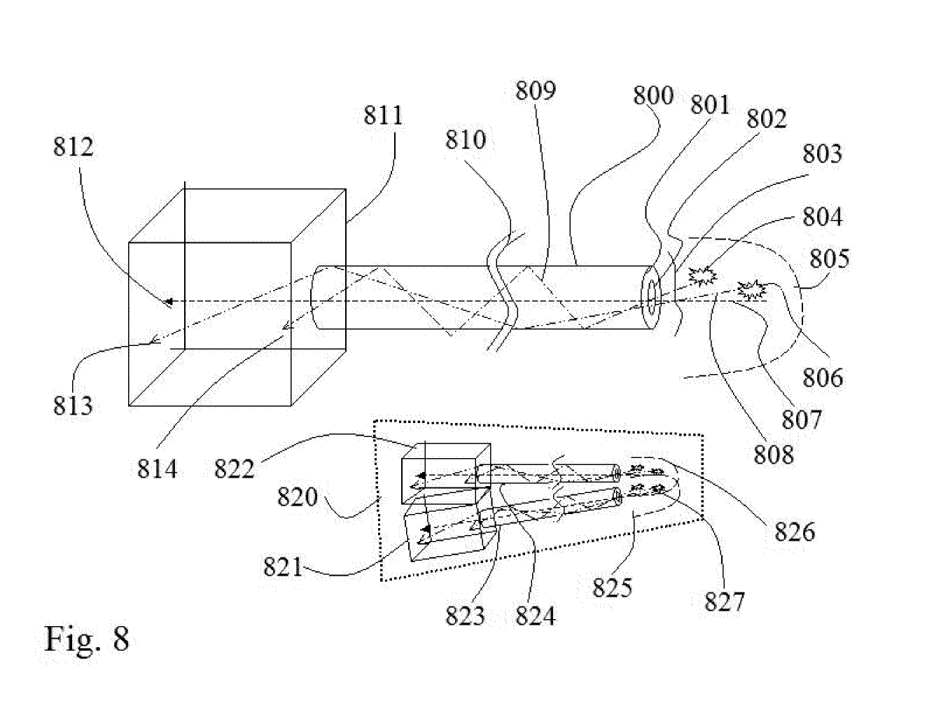

FIG. 8 shows details of the needle view that was previously discussed, but not detailed. Being a new method of imaging it might be required to be discussed in detail, being an embodiment of the present invention. It will be nice to place a CCD camera on the cutting tip, but this is not possible except with large needles with diameter over 4 mm, and that is what the current patent tries to avoid: making large wounds in the patient's body, for no substantial gain. Therefore the use of another imaging technology, based on computer image processing is the most appropriate. This technology may be good enough to serve as a microscope with magnifications under 100×, possibly eliminating further the need for pathologic in vitro analysis, because the image may be transmitted remotely to the specialist's location and make a diagnosis in real time. Various spectral bands may be also used as well as calibration tips. A calibration tip is a fine needle pushed by a capillary pressure actuator in the middle of the capillary array seen by all the imaging tube, that may have controlled movements used to calibrate the image reconstruction algorithms.

The imaging device module is made of a hollow or optic fiber capillary tube 800, that has an opening 802 that may be a pin-hole or a micro-optics array, fixed in a mount with hydrophobic deposition 801, that has the property of forming and holding the liquid interface 803 at some distance. Other lens and imaging devices at the tip might require the coating to be hydrophilic, in order to permit the best visualization of the nearby liquids and tissue parts 804, 806.

In the case of a pinhole optical fiber, the light reflected or emitted by the tissue or cells 804 is passing inside the capillary tube and reflecting inside 809 until it leaves the tube and hits a sensitive detector cell 814 inside the detector array 811. The light coming from the nearby molecule 806 is traveling on the path 808 until exits the capillary tube and hits the detector in 813. The central positioned objects emit on the path 807 and hit the detector cell in 812. Based on calibration the computer takes the signals and recomposes the image. The curve 805 shows the imaging field where depending on frequency used some attenuation is produced. The field depth may be considered at about 5 skin-depths inside the tissue, being usually in 1-5 mm range around the capillary tube that is shortened in 810 for clarity of illustration.

Assemblies of several such visualization modules may be hooked together forming a stereoscopic array of endoscopes 820 with one detection array 821 nearby another module 822, each having capillary tubes 823 and 824 and visualization volumes 825 and 826 that overlap, making 3D visualization possible in their intersection 827. In this way the operator through stereoscopic goggles can visualize a 3D virtual image reconstruction. This all may require a needle under 1 mm in diameter, and deliver the microscopic diagnosis too. Full chemical and drug sensitivity analysis of the targeted tissue may still require the biopsy sampling of tissue and in vitro processing.

### BRIEF DESCRIPTIONS OF INVENTION

The present invention refers to a set of improvements to the actual technique of tissue sampling for biopsy and tissue analysis using advanced spectrometry and capillary optical wave guides that brings the signal from the tissues, together with coordinates control to make the process minimally damaging. The proposed device makes the minimum wound possible to get the necessary information and to contain the bleeding and disease from spreading by using a complex plugging technique with various types of plugs. We also provide a chance for real time diagnosis, and immediate injection of drug treatment to the diseased tissue.

The main embodiment of the invention refers to the enhancement of the biopsy gun by adding a specialized notch that delivers a tissue plug simultaneously with taking the sample that replaces the missing tissue and helps healing. The sampler needle was equipped with two gamma sources for better tracking the device's coordinates inside the patient's body. The device may be also equipped with a set of capillary tubes used as wave guides in order to make real time spectrometric analysis in order to identify various diseased cells by their molecular signature. The needle may also carry sensors to identify the pH or other chemical/physical properties in the organ being sampled.

The new procedure will start with a very small penetration done by a needle that carries spectrometric and electro-chemical measurement capabilities being accompanied by several expandable cylindrical shells as presented in FIG. 4. After it reaches the area of interest and all the needed data have been obtained by spectral and electrochemical measurements, the central needle is withdrawn and the plugging needle presented in FIG. 5 is introduced.

If the gathered data from the first stage is not enough, a biopsy gun as presented in FIG. 3 is introduced, expanding the guiding shells, and the biopsy sample is taken simultaneously leaving the plug behind with a pretreatment identified at the previous step. After the biopsy gun is taken out, a plugging gun is introduced for plugging all the tissue boundaries along the entry path, and allowing the cylindrical tubing shells to be gradually withdrawn leaving the tissues behind plugged and with minimal damage.

The damage is also minimized by the small diameter insertion needle, which is followed by gradual stretching of the tissue as higher diameter guns are introduced in the cylindrical tubing shells. These tubing shells keep the diseased cells or tissues from spreading to other organs along the entry path. This invention develops a family of 3 different guns—for invasive analysis, sampling with replacement plug and plugging, and their combinations giving a variety of tools able to perform a better biopsy-sampling operation as shown in FIG. 6.

### EXAMPLES OF THE INVENTION

Thus it will be appreciated by those skilled in the art that the present invention is not restricted to the particular preferred embodiments described with reference to the drawings, and that variations can be made therein without departing from the scope of the present invention as defined in the appended claims thereof. The present invention consists in the development of a set of improved biopsy needles used for diagnosing accurately tissue localized disease inside the body of humans and animals, in customized versions, as gauge, length and functionalities.

The application of these customized versions will extend the range of usage minimizing the negative impact of the treatment on patients, and also reducing undesired collateral effects and medical complications. The use of the embedded sensors will bring a progress to medicine, allowing the patient body pressure, temperatures, flow, composition of the blood and its chemical properties to be monitored continuously and be used in diagnosis and equipment control. Some derivatives of this equipment, without the function of biopsy tissue sampling and extraction might be developed as tools for measurement purposes and plug application only. The application of the present teaching will generate a step forward in medicine, by intensively using multi-parameter monitoring and more body friendly invasive devices.

What is claimed is:

1. A sampling biopsy gun comprising:
a. A set of guiding tubes, shells for the inner devices,
b. Internal rod carrying the:
i. Sample notch and,
ii. Plug notch,
iii. Capillary tubes for optic radiation guiding,
iv. Capillary tubes to apply vacuum to hold the sample in the notch,
v. Capillary tubes for drug injection,
vi. Capillary tubes to apply pressure to remove the plug,
vii. Radioactive point sources for localization,
viii. Electrodes for pH measurement,
c. a set of thin protective cylindrical shells.

2. A sampling and analysis system comprising:
a. Penetration needle with attachments comprising:
i. a set of various gauge needles containing inside:
1. Capillary tubes for optical wave transport from a laser to tissue and from tissue to spectroscopic devices,
2. Capillary tubes for anesthetic and drug release,
3. Capillary tubes to apply pressure and vibration (sonicity) to activate cutting blade,
4. Radioactive sources for localization and imaging purposes,
5. Hydrophobic coating,
ii. A set of protective expandable gauge cylindrical shells covering the needles,
b. A sampler biopsy gun comprising:
i. An external shell,
ii. Internal rod carrying the:
1. Sample notch and,
2. Plug notch,
3. Capillary tubes to apply vacuum to hold the sample in the notch,
4. Capillary tubes for liquid drug injection,
5. Capillary tubes to apply pressure to remove the plug,
6. Radioactive X ray point sources for localization,
iii. Set of thin protective cylindrical shells,
c. A plugging gun comprising:
i. An external shell,
ii. Internal rod carrying the plugs,
iii. Radioactive sources for localization,
d. A set of cylindrical expandable gauge shells for bio-chemical protection.

3. A method of making invasive analysis and tissue sampling based on:
a. Smooth penetration using a small gauge needle carrying analytic capabilities,
b. Layers of shell-like tubings to protect the penetrated tissues from contamination,
c. IR-Vis spectroscopy to identify disease,
d. X-ray goniometry for device localization,
e. pH measurement,
f. Delivery of anesthetic and drugs,
g. Corroborate with imaging devices as ultrasound and CT,
h. enlarges the penetration hole by elastic stretching of the tissue,
i. uses positive pressure in the hole to prevent bleeding,
j. uses a sampler that plugs the hole left by the biopsy,
k. a plugging technology to isolate each penetrated organ,
l. expandable absorbable plugs impregnated with drugs,
m. US, CT, MRI and stereoscopic X ray imaging,
n. makes continuous recording of the process,
o. Compliant with ISO 14004 standard.

4. A sampler according claim 1 that uses vacuum to hold the tissue sample in the notch bed.

5. A sampler, according claim 1 that uses the hydrophobic coating to seal various sections.

6. A sampler according claim 2 that uses hollow capillary wave guides to transport the laser signals back from the tissue to detection sensors.

7. A method according claim 3 where the plug is designed to occupy the sample space and seal it in sectors localizing the bleeding and contamination.

8. A sampling and analysis system according to claim 2 where the three gamma goniometry units are used to accurately determine the position of the device.

9. A sampling and analysis system according to claim 2 where the outer sheathing and the tip of the needle has hydrophobic coating.

10. A sampling and analysis system according to claim 2 where the outer side of the needle has a coating with high ultrasound reflectivity.

11. A sampling and analysis system according to claim 2 where a laser light pulse is sent in the tissue to excite frequencies of the surrounding molecules to allow real time molecular disease identification.

12. A sampling and analysis system according to claim 2 where several hollow capillary tubes are used to simultaneously detect the molecular composition of the tissues.

13. A sampling biopsy gun according to claim 1 where the gun uses vacuum to extract the liquids from tissue for analysis and stabilize the tissue in the biopsy sample notch.

14. A sampling biopsy gun according to claim 1 where the gun carries and has the capability to apply various plugs as solid, gels, or liquids using the capillary tube for injection. Injected materials could be for hemostasis or drug treatment of the disease in the organ.

15. A sampling biopsy gun according to claim 1 where various gases and liquids are used via capillary tubes to apply positive pressure to increase the cito-hemo-static effect in the wounded areas and compensate for the negative pressure when the needle is removed inside the sheathing.

16. A sampling biopsy gun according to claim 1 where a set of consecutive expandable sheaths made of thin foils rolled cylindrically over the needle is used to shield and protect other penetrated tissues from being contaminated and maintain the same path in the body for higher diameter needles introduced successively performing sequential functions as optical spectral diagnosis, biopsy sampling and plugging, plugging and tubing withdrawal.

17. A method of making invasive analysis and tissue sampling according to claim 3 where the location of the device in the body is tracked by reference coordinates using various possible imaging systems.

18. A method of making invasive analysis and tissue biopsy sampling according the claim 3 with a gradual approach to minimize damage to the patient, starting with small diameter needles with sheathing, then if needed, using larger diameters for biopsy sampling, followed by plugging and gradual withdrawing after plugging the penetrated tissues' interfaces.

19. A method of making invasive analysis and tissue sampling according the claim 3 where empty capillary.

20. A method of making invasive analysis and tissue sampling according the claim 3 with plugging of the penetrated tissues by various gas, liquid or solid materials possibly containing drugs for hemostasis or treatment of the patient's underlying disease.
